(19) United States
(12) Patent Application Publication
(10) Pub. No.: US 2005/0154052 A1
(43) Pub. Date: Jul. 14, 2005

(54) Novel crystalline forms of (s)-citalopram oxalate

(75) Inventors: Reddy Bandi Parthasaradhi, Hyderabad (IN); Reddy Kura Rathnakar, Hyderabad (IN); Reddy Rapolu Raji, Hyderabad (IN); Reddy Dasari Muralidhara, Hyderabad (IN); Reddy Kesireddy Subash Chander, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Hetero Drugs Limited, Hydersbad, Andhrapradesh (IN)

(21) Appl. No.: 10/509,139

(22) PCT Filed: Mar. 24, 2003
(86) PCT No.: PCT/IN03/00066

(51) Int. Cl. A61K 31/343

(52) U.S. Cl. 514/469; 549/467

(57) ABSTRACT

The present invention relates to novel crystalline forms of (S)-citalopram oxalate, to processes for their preparation and to pharmaceutical compositions containing them.

### FIELD OF THE INVENTION

The present invention relates to novel crystalline forms of (S)-citalopram oxalate, to processes for their preparation and to pharmaceutical compositions containing them.

### BACKGROUND OF THE INVENTION

(S)-Citalopram of formula (1): 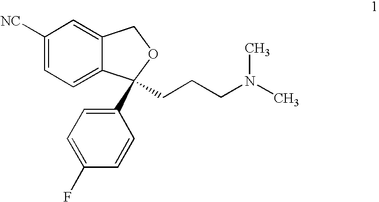
or 1(S)-[3-(Dimethylamino)propyl]-1-(4-fluorophenyl)-1,3-dihydro-5-isobenzofurancarbonitrile as well as acid addition salts thereof are valuable antidepressants. EP 0347066 disclosed the therapeutic uses of (S)-citalopram and its salts. In the prior art literature no crystalline forms of (S)-citalopram oxalate were reported

We have discovered two novel crystalline forms of (S)-citalopram oxalate. The novel forms have been found to be stable and reproducible and suitable for pharmaceutical preparations.

Thus the object of the present invention is to provide stable novel crystalline forms of (S)-citalopram oxalate, processes for preparation of the novel crystalline forms and pharmaceutical compositions containing these novel crystalline forms.

### DESCRIPTION OF THE INVENTION

According to one aspect of the present invention, there is provided a novel crystalline form of (S)-citalopram oxalate, designated as Form I, characterized by an x-ray powder diffraction pattern having peaks expressed as 2θ at about 6.9, 8.9, 10.8, 13.4, 14.0, 16.3, 17.6, 18.6, 19.1, 19.5, 21.2, 22.8, 23.1, 24.2, 24.5, 25.3, 27.3 degrees. FIG. 1 shows typical Form I x-ray powder diffraction pattern.

According to another aspect of the present invention, there is provided a process for preparation of Form I of (S)-citalopram oxalate. Thus, (S)-citalopram oxalate is mixed with a suitable solvent. The suitable solvent is selected from the group consisting of acetone, ethyl acetate, methyl tert-butyl ether, dioxane and acetonitrile. (S)-Citalopram oxalate prepared by the process described in, for example, EP 0347066 or Form II of (S)-citalopram oxalate (prepared by the process described below) may be used. The contents may be heated to reflux. The Form I of (S)-citalopram oxalate is separated by filtration.

According to another aspect of the present invention, there is provided an alternative process for the preparation of Form I of (S)-citalopram oxalate. Thus, (S)-citalopram is dissolved in a suitable solvent and oxalic acid is added to the solution. The suitable solvent is selected from the group consisting of acetone, ethyl acetate, methyl tert-butyl ether, dioxane and acetonitrile. The Form I of (S)-citalopram oxalate is precipitated from the solution by the techniques such as cooling, partial removal of the solvent or addition of anti-solvent.

According to one aspect of the present invention, there is provided a novel crystalline form of (S)-citalopram oxalate, designated as Form II, characterized by an x-ray powder diffraction pattern having peaks expressed as 2θ at about 6.6, 10.0, 11.0, 11.9, 15.2, 16.8, 17.8, 20.3, 21.1, 21.4, 22.6, 23.0, 26.4, 28.4 degrees. FIG. 2 shows typical Form II x-ray powder diffraction pattern.

According to another aspect of the present invention there is provided a process for preparation of the Form II of (S)-citalopram oxalate. Thus (S)-citalopram oxalate is mixed with an alcohol. (S)-Citalopram oxalate prepared by the process described in, for example, EP 0347066 or the Form I of (S)-citalopram oxalate may be used. The alcohol is either methanol or ethanol or isopropyl alcohol. The solubility of (S)-citalopram oxalate depends on the alcohol used and volume of the alcohol to (S)-citalopram oxalate. For example, 5 gm of (S)-citalopram oxalate is soluble in 35 ml of methanol at 25° C. If (S)-citalopram oxalate is soluble in the conditions of experiment, the Form II of (S)-citalopram oxalate is precipitated from the solution. The techniques such as cooling, partial removal of the solvent, addition of anti-solvent like diisopropyl ether may be used to precipitate the Form II of (S)-citalopram oxalate. If the (S)-citalopram oxalate is insoluble in the alcohol, after mixing (S)-citalopram oxalate and the alcohol the solid is filtered from the contents to obtain Form II of (S)-citalopram oxalate.

According to another aspect of the present invention, there is provided an alternative process for the preparation of Form I of (S)-citalopram oxalate. Thus, (S)-citalopram is dissolved in an alcoholic solvent and oxalic acid is added to the solution. The alcoholic solvent is either methanol or ethanol or isopropyl alcohol. (S)-citalopram prepared by the process described in, for example, EP 0347066 may be used. The Form II of (S)-citalopram oxalate is precipitated from the solution by the techniques such as partial removal of the solvent or addition of anti-solvent.

According to another aspect of the present invention there is provided a pharmaceutical composition comprising Form I or Form II of (S)-citalopram oxalate. The forms of (S)-citalopram oxalate may be formulated in a form suitable for oral administration or injection.

### BRIEF DESCRIPTION OF THE DRAWINGS

FIG. 1 is a x-ray powder diffraction pattern of Form I (S)-citalopram oxalate.

FIG. 2 is a x-ray powder diffraction pattern of Form II (S)-citalopram oxalate.

x-Ray powder diffraction spectrum was measured on a Siemens D5000 x-ray powder diffractometer having a copper-Kα radiation.

The following examples are given for the purpose of illustrating the present invention and should not be considered as limitations on the scope or spirit of the invention.

### EXAMPLE 1

(S)-Citalopram oxalate (5 gm, obtained as in example 2 of EP 0347066) is mixed with acetone (30 ml), heated to reflux and is cooled to 20° C. The separated crystals are filtered and dried to give Form I of (S)-citalopram oxalate (4.5 gm).

### EXAMPLE 2

(S)-Citalopram (10 gm, obtained as in example 2 of EP 0347066) is dissolved in acetone (100 ml) and oxalic acid dihydrate (5 gm) is added to the solution. The contents are maintained for 30 minutes at 0° C. and the separated solid is filtered and dried to give Form I of (S)-citalopram oxalate (10.5 gm).

### EXAMPLE 3

(S)-Citalopram oxalate(5 gm, obtained as in example 2 of EP 0347066) is dissolved in methanol (35 ml) at 25° C. Then diisopropyl ether (50 ml) is added to the solution and maintained for 2 hours at 25° C. The separated crystals are filtered and dried to give Form II of (S)-citalopram oxalate (4 gm).

### EXAMPLE 4

(S)-Citalopram (10 gm, obtained as in example 2 of EP 0347066) is dissolved in isopropyl alcohol (125 ml) and oxalic acid dihydrate (5 gm) is added to the solution. The contents are maintained for 30 minutes at 40° C. and cooled to 0° C. The separated solid is filtered and dried to give Form II of (S)-citalopram oxalate (9.5 gm).

### EXAMPLE 5

Example 1 is repeated using Form II of (S)-citalopram oxalate instead of (S)-citalopram oxalate (obtained as in example 2 of EP 0347066) to give Form I of (S)-citalopram oxalate.

### EXAMPLE 6

Example 3 is repeated using Form I of (S)-citalopram oxalate instead of (S)-citalopram oxalate (obtained as in example 2 of EP 0347066) to give Form II of (S)-citalopram oxalate.

1. A crystalline Form I of (S)-citalopram oxalate, characterized by an x-ray powder diffraction pattern having peaks expressed as 2θ at about 6.9, 8.9, 10.8, 13.4, 14.0, 16.3, 17.6, 18.6, 19.1, 19.5, 21.2, 22.8, 23.1, 24.2, 24.5, 25.3, 27.3 degrees.

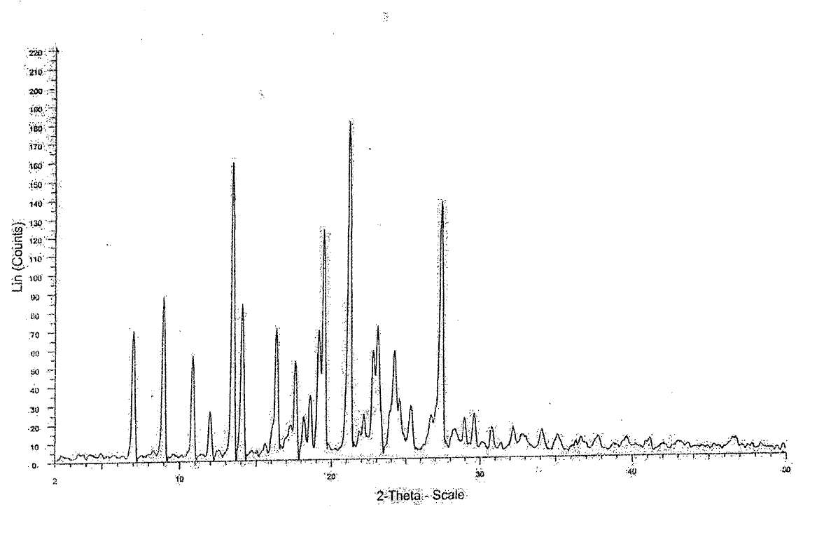

2. A crystalline Form I of (S)-citalopram oxalate as defined in claim 1, further characterized by an x-ray powder diffraction pattern as in FIG. 1.

3. A process for preparation of Form I of (S)-citalopram oxalate as defined in claim 1, which comprises:
a) mixing (S)-citalopram oxalate and a suitable solvent; and
b) isolating Form I of (S)-citalopram oxalate;
wherein the suitable solvent is selected from the group consisting of acetone, ethyl acetate, methyl tert-butyl ether and acetonitrile.

4. A process according to claim 3, wherein the suitable solvent is acetone.

5. A process according to claim 3, wherein the suitable solvent is ethyl acetate.

6. A process for preparation of Form I of (S)-citalopram oxalate as defined in claim 1, which comprises:
a) adding oxalic acid to a solution of (S)-citalopram in a suitable solvent;
b) isolating Form I of (S)-citalopram oxalate;
wherein the suitable solvent is selected from the group consisting of acetone, ethyl acetate, methyl tert-butyl ether and acetonitrile.

7. A process according to claim 6, wherein the suitable solvent is acetone.

8. A crystalline Form II of (S)-citalopram oxalate, characterized by an x-ray powder diffraction pattern having peaks expressed as 2θ at about 6.6, 10.0, 11.0, 11.9, 15.2, 16.8, 17.8, 20.3, 21.1, 21.4, 22.6, 23.0, 26.4, 28.4 degrees.

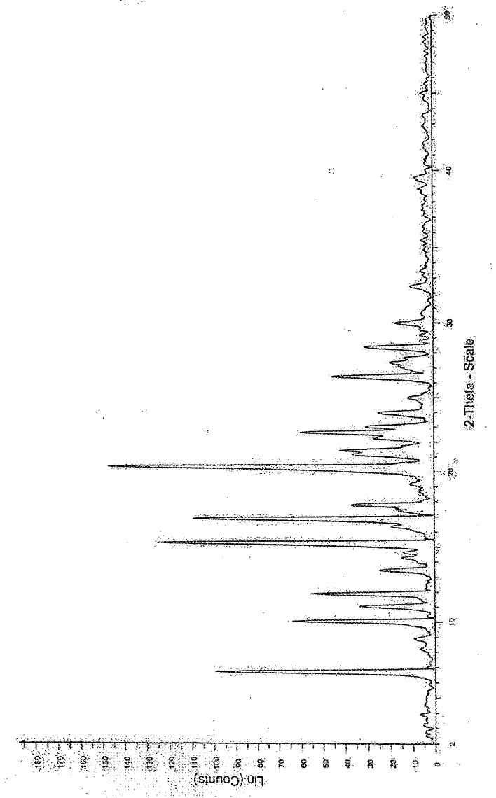

9. A crystalline Form II of (S)-citalopram oxalate as defined in claim 8, characterized by an x-ray powder diffraction pattern as in FIG. 2.

10. A process for preparation of Form II of (S)-citalopram oxalate as defined in claim 8, which comprises:
a) mixing (S)-citalopram oxalate and an alcoholic solvent;
b) isolating Form II of (S)-citalopram oxalate;
wherein the alcoholic solvent is selected from the group consisting of methanol, ethanol and isopropyl alcohol.

11. A process according to claim 10, wherein the alcoholic solvent is methanol.

12. A process according to claim 11, wherein Form II of (S)-citalopram oxalate is isolated by using diisopropyl ether as an anti-solvent.

13. A process for preparation of Form II of (S)-citalopram oxalate as defined in claim 8, which comprises:
a) adding oxalic acid to a solution of (S)-citalopram in an alcoholic solvent;
b) isolating Form II of (S)-citalopram oxalate;
wherein the alcoholic solvent is selected from the group consisting of methanol, ethanol and isopropyl alcohol.

14. A process according to claim 13, wherein the alcoholic solvent is methanol.

15. A pharmaceutical composition comprising the crystalline Form I of (S)-citalopram oxalate as defined in claim 1 and a pharmaceutically acceptable carrier.

16. A pharmaceutical composition comprising the crystalline Form II of (S)-citalopram oxalate as defined in claim 8 and a pharmaceutically acceptable carrier.
